(19) United States
(12) Patent Application Publication
(10) Pub. No.: US 2001/0053926 A1
(43) Pub. Date: Dec. 20, 2001

(54) Combined frequency-domain time-domain multiplexing of radio frequency communications with multiple implanted devices

(76) Inventor: Todd K. Whitehurst, Sherman Oaks, CA (US)

Correspondence Address: ALFRED E. MANN FOUNDATION FOR, SCIENTIFIC RESEARCH, PO BOX 905, SANTA CLARITA, CA, 91380, US

(21) Appl. No.: 09/882,521

(22) Filed: Jun. 15, 2001

Related U.S. Application Data
(60) Provisional application No. 60/212,854, filed on Jun. 20, 2000.

(51) Int. Cl. A61N 1/36

(52) U.S. Cl. 607/61

(57) ABSTRACT

A Functional Electrical Stimulation (FES) system provides frequency division multiplexed transmission of control signals from a master controller to a multiplicity of microstimulators. FES systems utilize the multiplicity of microstimulators to provide electrical signals to stimulate nerves and muscles to provide movement for paraplegics and quadriplegics. Each of the microstimulators are assigned to one of a multiplicity of carrier frequencies for receiving commands from the master controller. When a movement is desired, the microstimulator commands are modulated at the assigned carrier frequency for each microstimulator. The modulated signals for all of the microstimulators are combined into a single main carrier signal which is transmitted over an RF link to all of the microstimulators. Each microstimulator receives the main carrier signal, and filters the main carrier signal to recover the command for the microstimulator.

This application claims the benefit of U.S. Provisional Application Ser. No. 60/212,854, filed Jun. 20, 2000.

### BACKGROUND OF THE INVENTION

The present invention relates to Functional Electrical Stimulation (FES) systems and more particularly to an improvement to known methods for providing control signals to implanted microstimulators of an FES system. Such control signals are required at a high update rate to provide effective stable control of a patient's movement. The improvement provided by the present invention allows for a high effective update rate for a multiplicity of microstimulators while reducing the overall data transmission rate.

Paraplegics and quadriplegics often have muscles capable of functioning, but are paralyzed due to damage to nerves that carry impulses to the muscles. Additionally, individuals afflicted with neuro degenerative diseases such as polio and Amyotrophic Lateral Sclerosis (also known as ALS, or Lou Gehrig's disease) may be similarly disabled. Functional Electrical Stimulation provides such individuals with use of their muscles by providing artificial stimulation pulses to the patient's muscles, which stimulation pulses result in a desired movement.

Prosthetic devices have been used for some time to provide electrical stimulation to excite muscle, nerve or other cells. Some of these devices have been large bulky systems providing electrical pulses through conductors extending through the skin. Disadvantageously, complications, including the possibility of infection, arise in the use of stimulators which have conductors extending through the skin.

Other smaller stimulators are implants which are controlled through high-frequency, modulated, RF telemetry signals. An FES system using telemetry signals is set forth in U.S. Pat. No. 4,524,774, issued Jun. 25, 1985 for “Apparatus and Method for the Stimulation of a Human Muscle.” The '774 patent teaches a source of electrical energy, modulated by desired control information, to selectively control and drive numerous, small stimulators, disposed at various locations within the body. Thus, for example, a desired progressive muscular stimulation may be achieved through the successive or simultaneous stimulation of numerous stimulators, directed by a single source of information and energy outside the body.

Many difficulties arise in designing RF powered implantable stimulators which are small in size, and are also capable of receiving sufficient energy and control information to satisfactorily operate them without direct connection. A design of a small functionally suitable stimulator, a microstimulator, is taught is U.S. Pat. No. 5,324,316 issued Jun. 28, 1994 for “Implantable Microstimulator.” The '316 patent teaches all the elements required for successful construction and operation of a microstimulator. The microstimulator is capable of receiving and storing sufficient energy to provide the desired stimulating pulses, and also, is able to respond to received control information as to pulse duration, current amplitude and shape. Further, the stimulator of the '316 patent achieves a “charge balancing”, that is, a balancing of current flow through the body tissue in both directions to prevent damage to the tissue which results from continued, preponderance of current flow in one direction. The microstimulator of the '316 patent can also be easily implanted, such as by expulsion through a hypodermic needle. The '316 patent in incorporated herein by reference.

The microstimulator of the '316 patent requires a control signal having parameters that define the amplitude, width, and frequency of the desired stimulation pulse. In order to achieve effective functional control of muscles to move the patient's body in a desired manner, a pulse parameter update rate of as high as every 10 milliseconds is required. Additionally, a plurality of microstimulators may be required to stimulate different nerves or muscles in a coordinated manner to achieve the desired motion.

In some applications, one thousand or more microstimulators may be implanted in a single patient. Communicating with one thousand microstimulators at a 10 millisecond update rate requires one hundred thousand commands per second. Each command may contain as many as one hundred bits, resulting in a data rate of ten million bits per second, or ten mega bits per second. The resulting signal is 100 nanoseconds per bit. To ensure that the signal is successfully transmitted, the carrier signal must have several cycles per bit. The resulting transmit frequency is approaching one gigahertz, at which frequency the body absorbs RF energy and tissue damage is possible.

Additionally, the requirements for transmission of the signals by a master controller, and receipt and processing of the signals by a multiplicity of microstimulators, at very high data rates, results in increased power consumption and potentially high error rates.

What is therefore needed is a method for reducing the overall data transmission rate required to achieve effective functional control of the patient's muscles.

### SUMMARY OF THE INVENTION

The present invention addresses the above and other needs by providing a Functional Electrical Stimulation (FES) system utilizing radio frequency (RF) communications, which communications combine frequency division multiplexing and time division multiplexing, to communicate between a master controller and a multiplicity of implanted microstimulators. FES systems utilize the multiplicity of microstimulators to provide electrical signals to stimulate nerves and muscles to provide movement for paraplegics and quadriplegics. Each of the microstimulators are assigned to one of a multiplicity of carrier frequencies for receiving commands from the master controller. When a movement is desired, a set of microstimulators required for the movement is selected, and the stimulation level for each member of the set of microstimulator, are computed at up to a 100 Hz update rate. The microstimulator commands are transformed into modulated signals at the assigned carrier frequency for the microstimulator. The modulated signals for all of the microstimulators are combined into a single main carrier signal which is transmitted over an RF link to all of the microstimulators. Such a main carrier signal need only be transmitted at the frequency required for the highest frequency signal it contains, and all other signals comprising the main carrier signal essentially receive a free ride. Each microstimulator receives the main carrier signal, and filters the main carrier signal to recover the command for the microstimulator. Frequency division multiplexing control signals thus achieves a high update rate for nerve and muscle stimulation at a reduced data transmission rate.

In accordance with one aspect of the invention, there is provided a capability to transmit control signals to a multiplicity of microstimulators at a high command update rate without requiring a high data transfer rate. By frequency multiplexing the microstimulator commands over a multiplicity of sub bands, the effective data transfer rate is reduced to the rate within each sub band.

It is a feature of the invention that each of the multiplicity of microstimulators is only required to process signals within a single sub band. Thus, each microstimulator only requires a single band pass filter to extract the signal of interest from the multiplexed signal.

It is a further feature that the multiplicity of microstimulators may back transmit system parameters to the master controller. When such back transmission is exercised, each of the multiplicity of microstimulators may be assigned a second carrier frequency to transmit over. The master controller receives the back transmitted signals, and filters the signal to recover the individual signal.

It is an additional feature of the invention to assign a single sub band to a family of microstimulators. Such a family of microstimulators is a sub set of the multiplicity of microstimulators implanted in a patient. Further, the family of microstimulators may form a stimulation group, which stimulation group stimulates a muscle group, which muscle group is intended to work together to perform a task.

### BRIEF DESCRIPTION OF THE DRAWINGS

The above and other aspects, features and advantages of the present invention will be more apparent from the following more particular description thereof, presented in conjunction with the following drawings wherein:

FIG. 1 depicts a master controller and families of microstimulators controlled by the master controller;

FIG. 2 shows a multiplicity of microstimulators within a typical muscle;

FIG. 3 depicts a functional flow diagram for processing within a master controller controlling the multiplicity of microstimulators, using frequency multiplexed communications; and

FIG. 4 shows a functional flow diagram for processing within a multiplicity of microstimulators receiving control signals from a master controller, using frequency division multiplexing.

Corresponding reference characters indicate corresponding components throughout the several views of the drawings.

### DETAILED DESCRIPTION OF THE PREFERRED EMBODIMENTS

The following description is of the best mode presently contemplated for carrying out the invention. This description is not to be taken in a limiting sense, but is made merely for the purpose of describing the general principles of the invention. The scope of the invention should be determined with reference to the claims.

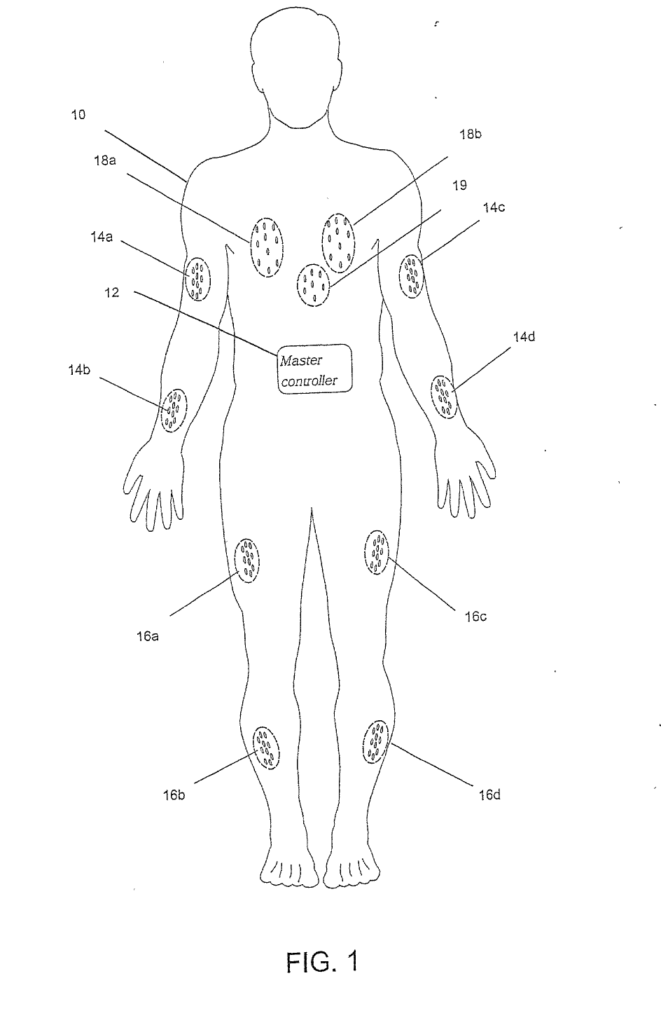

A Functional Electrical Stimulation (FES) system generates electrical signals to stimulate nerves and muscles to provide movement for paraplegics, quadriplegics, and other individuals with a neural lesion or a neuro degenerative disease. FIG. 1 shows a master controller 12 of an FES system centrally carried on a patient 10. The master controller 12 transmits control signals to a multiplicity of microstimulators. For example, the multiplicity of microstimulators may include: microstimulator arm groups 14a-14d, microstimulators leg groups 16a-16d, microstimulator lung groups 18a, 18b, and a microstimulator heart group 19.

As can be seen from FIG. 1, in cases where neuro degenerative disease has severely disrupted the bodies ability to control muscles, the number of microstimulators may grow extremely large in order to provide control for both motion of the arms and legs, and for heart, lung, and other organ functions.

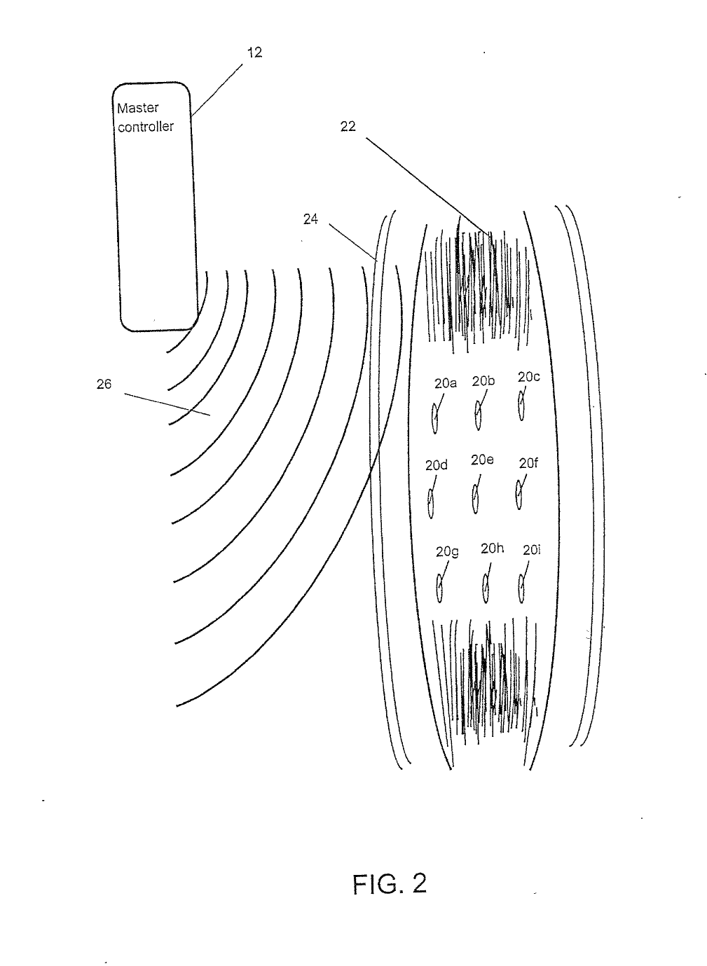

A view of microstimulators 20a- 20i implanted in a muscle 22 of the patient 10 is shown in FIG. 2. The microstimulators 20a- 20i are separated from the master controller 12 by skin 24 of the patient 10. U.S. Pat. No. 5,324,316 issued Jun. 28, 1994 for “Implantable Microstimulator” teaches the elements required for successful construction and operation of a microstimulator. Such microstimulator advantageously may be implanted through a large gauge needle and provides a minimally invasive implant procedure. The master controller 12 provides control signals to the microstimulators 20a- 20i using an RF signal 26. The set comprising all of the microstimulators implanted in a patient 10 will hereafter be referred to as a multiplicity of microstimulators 20a, 20b, . . . 20n.

However, the small size of the microstimulators requires that both the antenna for receiving the command signals, and the internal circuits for processing command signals, be as small and as low power as possible. Such size and power requirements conflict with functional requirements for high update rates for stimulation parameters, which update rates are needed to obtain stable and effective artificial control of muscles. Known communications systems utilized frequency division multiplexing to provide transmission of a multiplicity of signals over a single main channel. Individual signals are carried in non-overlapping subchannels within the main channel. Using frequency division multiplexing, the master controller may therefore provide control signals to the multiplicity of microstimulators 20a, 20b, . . . 20n wherein the effective data receipt rate of each of the multiplicity of microstimulators 20a, 20b, . . . 20n is substantially reduced.

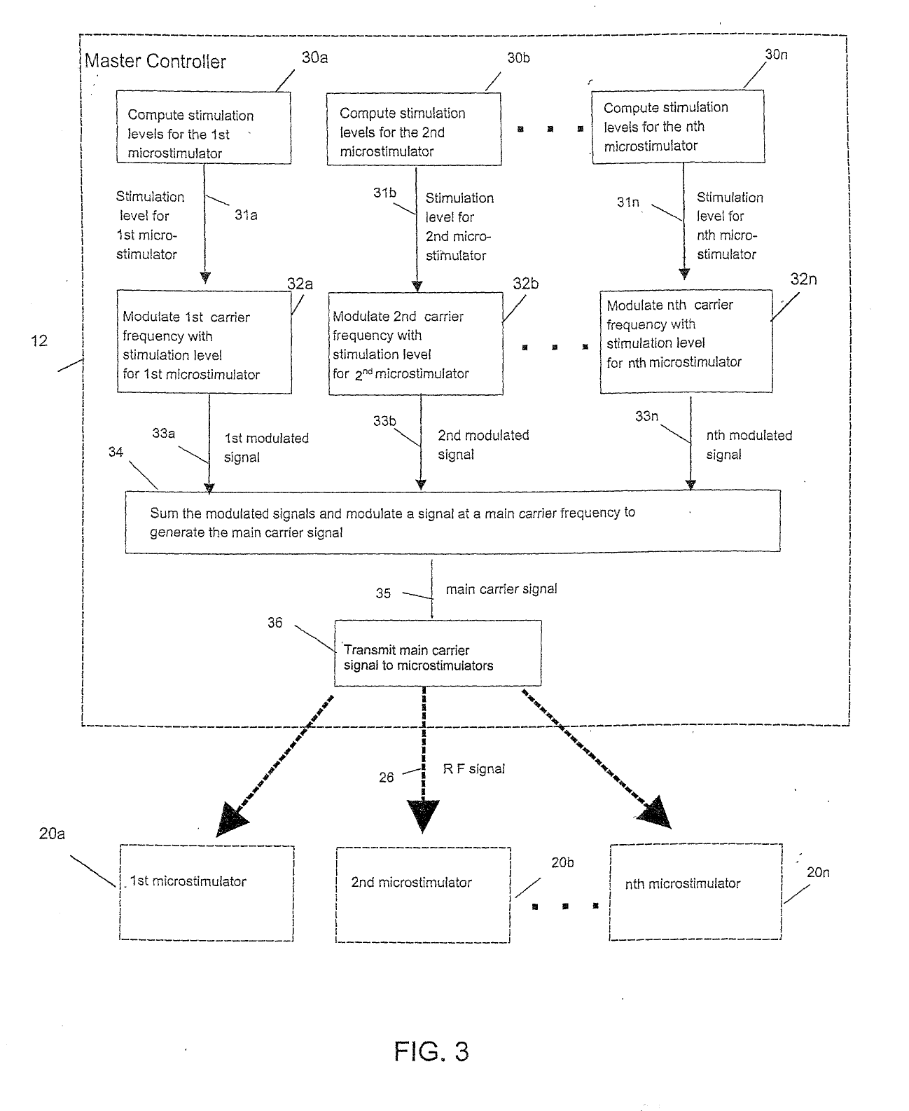

A functional flow diagram for communicating between a master controller 12 and the multiplicity of microstimulators 20a, 20b, . . . 20n, using frequency division multiplexing, is shown in FIG. 3. Compute stimulation level modules 30a, 30b, . . . 30n compute respective stimulation levels 31a, 31b, . . . 31n required for each of the multiplicity of microstimulators 20a, 20b, . . . 20n to achieve a desired result. That result may be a movement of a limb, a contraction of a lung muscle to cause the patient to breath, or any other muscle contraction utilized to achieve the desired result. A carrier frequency is assigned to each of the plurality of microstimulators. Each of the carrier frequencies may be assigned to a single microstimulator, or assigned to several microstimulators. The stimulation levels 31a, 31b, . . . 31n are provided respectively to modulate carrier frequency modules 32a, 32b, . . . 32n where modulated signals 33a, 33b, . . . 33n are computed by modulating the assigned frequency for each of the multiplicity of microstimulators 20a, 20b, . . . 20n with the stimulation level computed for the microstimulator. The modulated signals 33a, 33b, . . . 33n are then provided to the respective sum modulated signals module 34 wherein the modulated signals 33a, 33b, . . . 33n are summed to form a main carrier signal 35. The main carrier signal 35 is then provided to the transmit main carrier signal module 36 where it is transmitted as an RF signal 26 to the multiplicity of microstimulators 20a, 20b, . . . 20n.

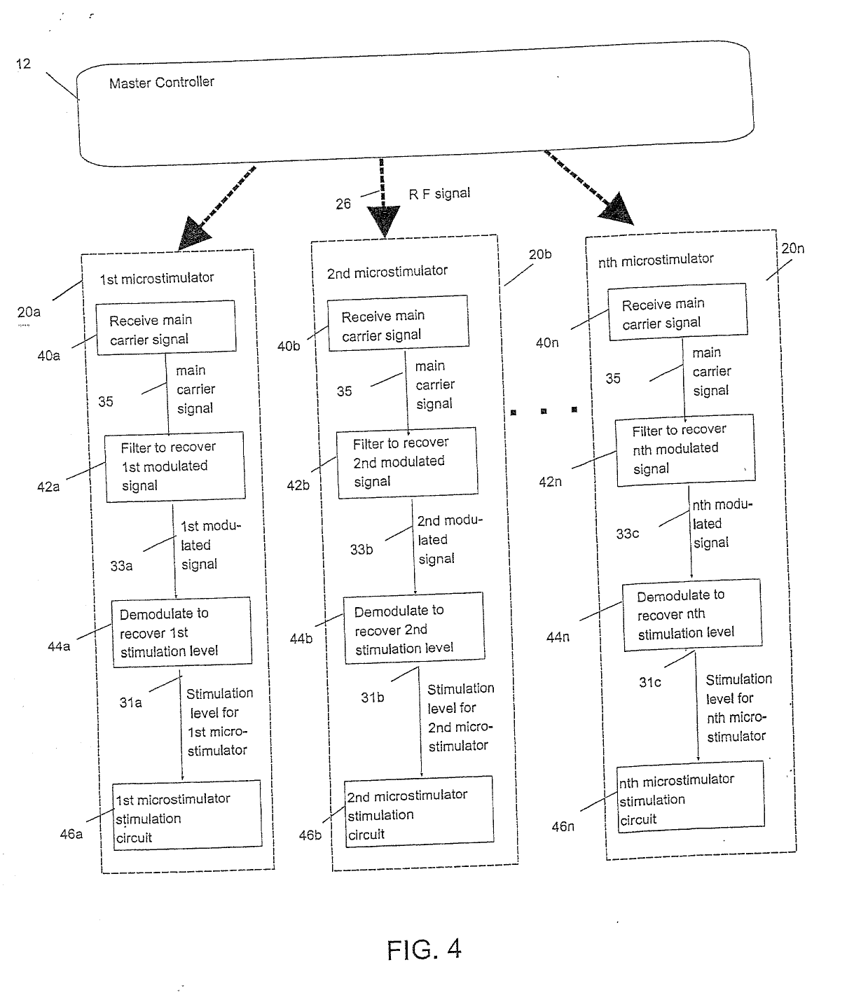

Each of the multiplicity of microstimulators 20a, 20b, . . . 20n receives the RF signal 26, as shown in FIG. 4. The RF signal 26 is received by receive main carrier signal modules 40a, 40b, . . . 40n. The receive main carrier signal modules 40a, 40b, . . . 40n include an antenna and any associated electronics required to receive the RF signal 26 and to output the main carrier signal 35 that is transmitted. The main carrier signal 35 is provided to filter to recover modulated signal modules 42a, 42b, . . . 42n. The filter to recover modulated signal modules 42a, 42b, . . . 42n include band pass filters with pass bands selected to pass the modulated signal modulated by the control signal intended for the microstimulator performing the filtering. The modulated signals 33a, 33b, . . . 33n that result from filtering are provided respectively to demodulate to recover stimulation level modules 44a, 44b, . . . 44n. The demodulate to recover stimulation level modules 44a, 44b, . . . 44n demodulate the respective signals to recover the stimulation levels 31a, 31b, . . . 31n computed in the master controller 12. The stimulation levels 31a, 31b, . . . 31n are provided to respective microstimulator stimulation circuits 46a, 46b, . . . 46c. The microstimulator stimulation circuits 46a, a6b, . . . 46c then apply the commanded stimulation levels 31a, 31b, . . . 31n to the patient 10.

In a manner similar to the above described frequency division multiplexing, the multiplicity of microstimulators 20a, 20b, . . . 20n may transmit messages to the master controller 12. The microstimulators may provide feedback to the master controller 12 of parameters regarding microstimulator performance, or the microstimulators may transmit the results of measurements of physiological conditions of the patient. For the purpose of transmitting signals from the multiplicity of microstimulators 20a, 20b, . . . 20n to the master controller 12, one of a plurality of second carrier frequencies is assigned to each of the multiplicity of microstimulators 20a, 20b, . . . 20n. One or more of the multiplicity of microstimulators 20a, 20b, . . . 20n may share the same second carrier frequency. The master controller 12 receives the signals from all of the multiplicity of microstimulators 20a, 20b, . . . 20n and processes the signal in the same manner as the multiplicity of microstimulators 20a, 20b, . . . 20n processed the RF signal 26 from the master controller 12, as described in FIG. 4.

Those skilled in the art will recognize that many variations of the embodiment described above exist, and these variations are contemplated by the present invention. For example, in an advanced embodiment, individual microstimulators may be reassigned to new carrier frequencies based on the nature of the commands that will immediately follow the reassignment. By making such periodic assignments, the use of carrier frequencies may be optimized. In such use, the band pass filter may be a digital implementation with stored coefficients available for each carrier frequency. In other embodiments, multiple master controllers may be used, which multiple master controllers control families of microstimulators.

In yet another embodiment, a single master controller may provide control signals for the multiplicity of microstimulators using a multiplicity of independent transmit modules. The sum the modulated signals module 34, and the transmit main carrier signal module 36, shown in FIG. 3, may be replaced by the multiplicity of independent transmit modules, which modules transmit respective modulated signal 33a, 33c, . . . 33n, wherein each of the multiplicity of independent transmit modules is dedicated to a single modulated signal. Advantageously, each of the multiplicity of independent transmit modules may include an antenna with a transmit pattern directed towards the microstimulators it controls.

While the invention herein disclosed has been described by means of specific embodiments and applications thereof, numerous modifications and variations could be made thereto by those skilled in the art without departing from the scope of the invention set forth in the claims.

What is claimed is:

1. A method for sending signals between a controller and a multiplicity of actuators comprising:
positioning a multiplicity of actuators, wherein the multiplicity of actuators perform a task, and wherein the multiplicity of actuators are positioned to facilitate performance of the task;
positioning a controller, wherein the controller communicates with the multiplicity of actuators, and wherein the controller is positioned to facilitate effective communication between the controller and the multiplicity of actuators;
assigning one of at least two carrier frequencies to each of the multiplicity of actuators;
obtaining at least two actuator signals in the controller;
modulating the least two carrier frequencies with the at least two actuator signals to generate at least two modulated signals;
combining the at least two modulated signals to generate a main carrier signal;
sending the main carrier signal from the controller to the multiplicity of actuators;
receiving the main carrier signal in the multiplicity of actuators; and
recovering the actuator signals from the main carrier signal.

2. The method of claim 1 wherein the multiplicity of actuators are a multiplicity of microstimulators, and wherein positioning a multiplicity of actuators comprises implanting the multiplicity of microstimulators within a patient.

3. The method of claim 2 wherein obtaining at least two actuator signals comprises computing a stimulation level required for each of the multiplicity of microstimulators to achieve a desired muscle contraction.

4. The method of claim 3 wherein modulating at least two carrier frequencies includes:
assigning a carrier frequency for each of the multiplicity of microstimulators; and
modulating the carrier frequency assigned to each of the multiplicity of microstimulators with the stimulation level computed for each of the multiplicity of microstimulators.

5. The method of claim 4 wherein combining the at least two modulated signals to generate a main carrier signal comprises:
summing the at least two modulated signals to generate a summed signal;
selecting a main carrier frequency; and
modulating the main carrier frequency with the summed signal to generate the main carrier signal.

6. The method of claim 5 wherein sending the main carrier signal comprises transmitting the main carrier signal over a radio frequency (RF) link between the controller and the multiplicity of microstimulators, and wherein receiving the main carrier signal comprises receiving the main carrier signal transmitted over the RF link with antenna in each of the multiplicity of microstimulators.

7. The method of claim 6 wherein recovering the actuator signals comprises:
filtering the main carrier signal to only pass the frequency band containing the modulated signal containing the stimulation level for the microstimulator; and
demodulating the modulated signal to obtain the stimulation level for the microstimulator.

8. The method of claim 1 further including:
generating microstimulator signals within the multiplicity of microstimulators;
assigning one of a multiplicity of second carrier signals to each of the multiplicity of microstimulators;
modulating the multiplicity of second carrier frequencies with the microstimulator signals to generate second modulated signals;
transmitting the second modulated signals from the multiplicity of actuators to the controller;
receiving the second modulated signals in the controller to generate a received signal;
filtering the received signal to recover the second modulated signals; and
demodulating the recover the microstimulator signals.

9. A Functional Electrical Stimulation (FES) system comprising:
a master controller; and
a multiplicity of microstimulators;
wherein the master controller provides control signals to the multiplicity of microstimulators using frequency division multiplexing.

10. The FES system of claim 9 wherein stimulation levels for each of the multiplicity of microstimulators are computed within the master controller.

11. The FES system of claim 10 wherein carrier frequencies are assigned to each of the multiplicity of microstimulators, and wherein the carrier frequency assigned to each of the multiplicity of microstimulators is modulated with the respective stimulation level computed for each of the multiplicity of microstimulators to generate a modulated signal for each of the multiplicity of microstimulators.

12. The FES system of claim 11 wherein the modulated signals are summed to form a main carrier signal, and wherein the main carrier signal is transmitted to the multiplicity of microstimulators as an RF signal.

13. The FES system of claim 12 wherein the RF signal is received by each of the multiplicity of microstimulators and the main carrier signal is generated.

14. The FES system of claim 13 wherein the main carrier signal is filtered using a band pass filter within the multiplicity of microstimulators to recover the respective modulated signal, wherein each bandpass filter is designed to pass the carrier frequency assigned to the respective microstimulator.

15. The FES system of claim 14 wherein the modulated signals are demodulated to recover the stimulation level for the respective microstimulator.

16. An implantable electrical stimulation and monitoring system comprising:
a controller; and
a multiplicity of microdevices;
wherein the controller computes control signals for the multiplicity of microdevices and provides the control signals to the multiplicity of microdevices using frequency division multiplexing, and wherein the multiplicity of microdevices measure physiological parameters of a patient and transmits the physiological parameters to the master controller.

17. The system of claim 16 wherein carrier frequencies are assigned to each of the multiplicity of microdevices, and wherein modulated signals are generated by using the control signals for each microdevice to modulate the respective carrier frequencies, and wherein a main carrier signal is generated by summing the modulated signals, and wherein the main carrier signal is transmitted to the multiplicity of microdevices.

18. The system of claim 17 wherein each of the multiplicity of microdevices receives the main carrier signal transmitted by the controller, and wherein each of the multiplicity of microdevices filters the main carrier signal with a bandpass filter designed to pass the carrier frequency assigned to the respective microdevice to recover the modulated signal and wherein the modulated signals are demodulated to recover the control signal for the respective microdevice.

19. The system of claim 18 wherein the main carrier signal is transmitted over an RF link.

20. The system of claim 19 wherein the microdevices may be implanted through the lumen of a large gauge needle.
